(19) United States
(12) Patent Application Publication
(10) Pub. No.: US 2003/0235630 A1
(43) Pub. Date: Dec. 25, 2003

(54) Dental hygiene products and methods of making dental hygiene products

(76) Inventor: Kenneth H. Nussen, Santa Monica, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 10/177,535

(22) Filed: Jun. 21, 2002

(51) Int. Cl. A61K 47/36; A61K 35/78

(52) U.S. Cl. 424/729; 514/777; 427/2.29

(57) ABSTRACT

A composition including green tea extract and wax. A carrier and a method of coating a carrier with a composition including green tea extract and wax, wherein the carrier is suitable for manipulation about at least one tooth of a user. An edible film containing green tea extract and adapted to dissolve in the mouth of a consumer. A method including mixing one or more film forming agent(s) with green tea extract (GTE) to form a composition; drying the composition on a substrate to form an edible film.

### BACKGROUND

1. Field

Dental hygiene and dental floss and edible dental film.

2. Background

An oral hygiene product incorporating tea tree oil can provide an antibacterial effect that may help to reduce bad breath and retard the development of plaque. Tea tree oil has several drawbacks, however, including its strong medicinal odor and its inadequacy at preventing inflammation of the gums. These factors have contributed to the unavailability of a tea tree oil oral hygiene product that is both pleasant to use and has anti-inflammatory properties.

Green tea extract is derived from the leaves of the plant Camellia sinensis and is rich in antioxidants. Green tea extract is commonly available in pill form as a nutritional supplement. Green tea extract has been shown to be effective at fighting cavities, prohibiting the growth of oral bacteria, and reducing inflammation of the gums.

### BRIEF DESCRIPTION OF THE DRAWINGS

The claims are illustrated by way of example and not by way of limitation in the figures of the accompanying drawings in which like references indicate similar elements. It should be noted that references to “an” or “one” embodiment in this disclosure are not necessarily to the same embodiment, and such references mean at least one.

FIG. 1 is a view of an unopened single use package of dental floss.

FIG. 2 is a view of an opened single use package of dental floss.

FIG. 3 is a view of a single use package of edible film.

FIG. 4 is a view of a multiple use package of edible film.

### DETAILED DESCRIPTION

Floss is a thread, ribbon, or tape typically made of Nylon or similar material that is used, in dental applications, to remove food particles and plaque from between the teeth and under the gums. Floss is used by inserting it between a pair of teeth and then pulling it back and forth across the lateral surface of each tooth. Floss is sold to the consumer in packages that typically contain a small spool of floss.

Floss is typically coated with wax. A wax coating allows the floss to more easily slide between the teeth. Some wax coatings contain other ingredients, such as cavity fighters or flavoring. Coatings may be applied to floss by running/pulling the floss through a vat holding a composition containing melted wax and other ingredients. The wax is usually heated to a high temperature to melt the wax, therefore any additional ingredients that are added must be able to withstand the heat without breaking down or becoming toxic. After exiting the vat, the floss is optionally put into consumer packaging.

In one embodiment, a floss, ribbon, or tape suitable for use in dental applications, such as maneuvering between adjacent teeth, describes a form or type of carrier. The carrier may include one or more of nylon, polytetrafluoroethylene (PTFE) (e.g., Teflon®), 840 Denier, or other similar materials. Coated on the carrier in one embodiment is a composition including wax and green tea extract (GTE). GTE has been shown to have anti-cavity, antibacterial, and anti-inflammatory properties when used as a dentifrice. In another embodiment, the composition includes wax, GTE, and tea tree oil (TTO). Other carrier types such as, but not limited to, plastic or wooden toothpicks having a similar composition coated thereon are also contemplated.

In one embodiment, the composition contains wax and up to two percent by volume (or 0.24 percent by weight) GTE. The wax is, for example, a bio-compatible wax such as a microcrystalline wax. In another embodiment, the wax or the composition may contain a flavorant (e.g., mint, cinnamon, bubble gum, anise, etc.) Some flavorants, such as anise, may have antiseptic and antibacterial qualities. The following table is an exemplary composition wherein GTE is combined with a flavorant (peppermint):

| Item | Dry Weight | Volume |
|---|---|---|
| GTE | 0.24% | 0.20% |
| Peppermint Oil | 25.64% | 24.80% |
| Microcrystalline Wax | 74.12% | 75.00% |

Amounts of GTE greater than two percent by volume may result in the formation of aggregates. In another embodiment, the composition may additionally contain 10 to 12 percent by volume (or up to 12.5 percent by weight) TTO. TTO has been shown to have natural antiseptic properties. Although TTO has a strong medicinal odor, this odor will tend to be masked by the presence of GTE in the composition. Thus, the amount of GTE is selected, in one embodiment, to sufficiently mask the medicinal odor of the TTO. It is appreciated that, in one instance, the medicinal odor of the TTO may still be present but that GTE renders the odor tolerable. In a composition including 11.8 percent by volume (or 12.5 percent by weight) TTO, an amount of two percent by volume (or 0.24 percent by weight) GTE has been determined to sufficiently mask the medicinal odor of the TTO rendering the product (carrier plus composition) satisfactory to consumers. The following table illustrates an exemplary composition of GTE and TTO:

| Item | Dry Weight | Volume |
|---|---|---|
| GTE | 0.24% | 0.20% |
| TTO | 12.50% | 11.80% |
| Microcrystalline Wax | 87.26% | 88.00% |

The composition may be formed by heating the wax to a temperature above the melting point. Freeze-dried GTE (up to two percent by volume) commercially available from XEL Herbaceuticals of Salt Lake City, Utah is added to the melted wax and the composition mixed (stirred). In an embodiment where a coating composition also includes TTO, the TTO (up to 12 percent by volume),commercially available from Natural Extracts of Australia of Los Angeles, Calif. is added as a liquid to the melted wax. One representative composition is 4 grams of GTE, 236 milliliters of TTO, and 1760 milliliters of microcrystalline wax. Both GTE and TTO in the proportions contemplated will not become toxic or break down when combined with the heated wax.

A carrier may be coated by dipping the carrier in the composition (the liquified composition). The coated carrier is then ambient cooled and placed into a package for distribution and use. Representative packaging includes, but is not limited to, a spool, a single use package, a vile, or a floss holder.

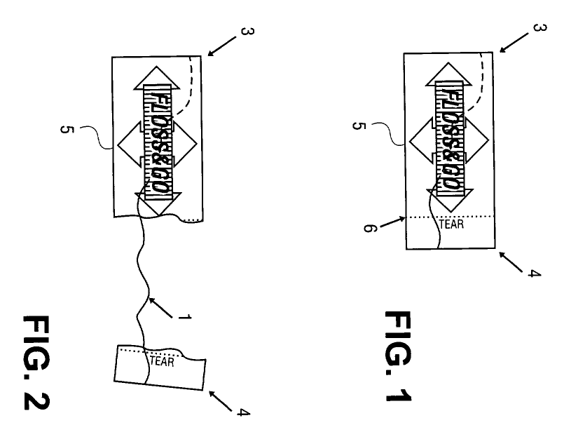

FIG. 1 is a view of an unopened single use package of dental floss in one embodiment. Package 5 contains a strand of floss (not shown) of sufficient length (e.g., on the order of about 10 to 20 inches) for a single use. Package 5 has a perforation 6 which allows the package to be torn such that portion 4 becomes separated from portion 3.

FIG. 2 is a view of the single use package of dental floss of FIG. 1 once the package 5 is opened, such as by tearing at perforation 6. In FIG. 2, Package 5 is separated in two such that portion 3 is separated from portion 4 and floss 1 is exposed. One end of floss 1 may be connected inside portion 3 and the other end of floss 1 may be connected inside portion 4, such as with a suitable adhesive, so that floss 1 stays with package 5. Alternatively, only one end (or neither end) of floss 1 is connected within a portion of package 5. By holding portion 3 and portion 4, floss 1 may be used to clean teeth. Package 5 (including portion 3 and portion 4) and floss 1 may then be discarded.

In another embodiment, effective amounts of GTE and TTO can be delivered orally in the form of an edible film that dissolves when it comes in contact with saliva. Edible film is a thin strip of edible material that is non-sticky to the touch and pliable, making it easy to package and use. When the film is placed in the mouth, it interacts with saliva and dissolves, releasing active ingredients. Edible film is sold to the consumer in packages that typically contain a number of small strips. One such product is the subject of a pending U.S. patent application Ser. No. 09/836,474, publication number US2001/0022964.

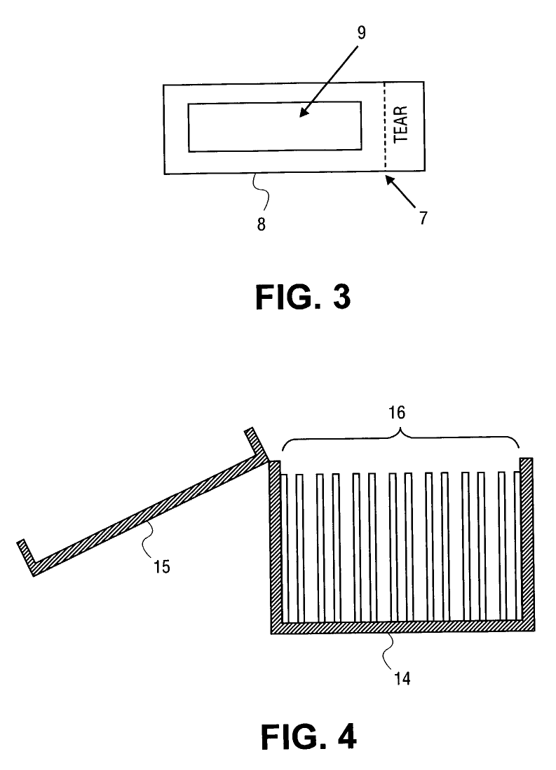

Edible film is made by combining one or more film forming agents with one or more active ingredients. The film composition may be heated to promote dissolution of the ingredients and subsequent drying. The film forming agents and the active ingredients must be in amounts that are non-toxic and safe for human consumption. In one embodiment, the active ingredients may be combined and optionally hydrated before being added to the film composition. The resulting film composition is then transferred to a suitable substrate where it dries to form a thin film. Drying can be accelerated by exposing the film composition to heated air or by heating the substrate. When dried, the film is cut into strips of suitable size and packaged. Representative packaging includes, but is not limited to, a spool, a single use package (FIG. 3), and a multiple use package (FIG. 4).

The film forming agents enable the film composition to dry into a pliable film without interfering with or diminishing the effectiveness of the active ingredients. In one embodiment, one or more film-forming agents such as pullulan, xanthan gum, locust bean gum, and carrageenan are combined with an active ingredient, such as an amount of GTE that is effective at reducing oral bacteria and fighting cavities. A suitable amount includes, but is not limited to, about two to five percent by weight GTE in a batch. In another embodiment, GTE is combined with TTO in an amount sufficient to mask the odor and taste of the TTO.

Flavoring (e.g., mint, cinnamon, bubble gum, anise, etc.), coloring, and sweeteners may optionally be added to the film composition. In one embodiment, one or more sweeteners such as saccharin, fructose, sucrose, maltose, and corn syrup are added to the film composition. The flavoring, coloring and sweeteners may be combined before being added to the film composition. In another embodiment, mint flavor is added to the film composition, for example, in an amount of approximately 10 percent by weight of the composition.

The film composition may be formed by hydrating (if necessary) one or more film forming agents to create a film composition. To the film composition is added freeze-dried GTE commercially available from XEL Herbaceuticals of Salt Lake City, Utah. In an embodiment where the edible film also includes TTO, TTO liquid commercially available from Natural extracts of Australia of Los Angeles, Calif. is added to the film composition.

After combining the ingredients, the film composition is then transferred onto a suitable substrate. A suitable substrate is nonporous and will not react chemically with the film composition. As the film composition dries on the substrate, it changes state from a liquid to a pliable film. The film is then cut into strips and packaged. The film is used by consumers in the following manner. A consumer will remove a piece of film from its packaging and place the film on their tongue. The film will dissolve as it comes in contact with saliva, thereby releasing active ingredients such as GTE and TTO.

FIG. 3 is a view of a single use package of edible film. The edible film 9 is contained within package 8. Package 8 can be opened by tearing along perforation 7. The film 9 is then removed from the package 8 and placed in the consumer's mouth were it will dissolve.

FIG. 4 is a view of a multiple use package of edible film. The package is comprised of body 14 and lid 15. Lid 15 may be detached from body 14. Inside body 14 are an array of edible strips 16, such that a consumer can carry a quantity of strips in a single container.

The specification has been described with reference to specific embodiments thereof. It will, however be evident that various modifications and changes can be made thereto without departing from the broader spirit and scope of the appended claims. The specification and drawings are, accordingly, to be regarded in an illustrative rather than a restrictive sense.

What is claimed is:

1. An apparatus comprising:
a carrier suitable for manipulation about at least one tooth of a user, the carrier coated in a composition comprising green tea extract (GTE) and wax; and
a vehicle to hold the carrier.

2. The apparatus of claim 1 further comprising:
tea tree oil (TTO).

3. The apparatus of claim 1, wherein GTE is present in an amount up to 0.2 percent by volume of the composition.

4. The apparatus of claim 2, wherein TTO is present in an amount up to 11.8 percent by volume of the composition.

5. The apparatus of claim 1, wherein the carrier is one of floss, ribbon, and tape.

6. The apparatus of claim 5, wherein the carrier is comprised of a material containing at least one of nylon, polytetrafluoroethylene, and 840 Denier.

7. The apparatus of claim 1, wherein the wax is flavored.

8. The apparatus of claim 7, wherein the wax flavor is one of mint, cinnamon, anise, and bubble gum.

9. The apparatus of claim 1, wherein the vehicle is one of a spool, a single use package, a vial, and a floss holder.

10. A method comprising:
forming a composition comprising green tea extract (GTE) and wax;
coating a carrier with the composition wherein the carrier is suitable for manipulation about at least one tooth of a user; and
placing the coated carrier into a vehicle.

11. The method of claim 10, wherein the composition further comprises:
tea tree oil (TTO).

12. The method of claim 10, wherein the carrier is one of floss, ribbon, and tape.

13. A composition comprising green tea extract (GTE) in an edible film adapted to dissolve in the presence of saliva within a mouth of a consumer.

14. The composition of claim 13, comprising tea tree oil (TTO).

15. A method comprising:
mixing one or more film forming agent(s) with green tea extract (GTE) to form a composition;
drying the composition on a substrate to form an edible film.

16. The method of claim 15, wherein the one or more film forming agents are selected from the group consisting of pullulan, xanthan gum, locust bean gum, and carrageenan.

17. The method of claim 15, further comprising:
cutting the edible film into strips suitable for packaging.

18. The method of claim 17, further comprising:
packaging the edible film strips, wherein the package is one of a spool, a single use package, and a dispenser for pre-cut strips.

19. An edible film produced according to the method of claim 15.

20. A method of delivering an anti-cavity and anti-inflammatory ingredients by introducing the edible film of claim 15 into an oral cavity.
